(19) United States
(12) Patent Application Publication
(10) Pub. No.: US 2026/0191677 A1
(43) Pub. Date: Jul. 9, 2026

(54) MANDIBLE ADJUSTMENT APPARATUSES

(71) Applicant: Shinrin-Yoku Traders LLC, Sherwood, OR (US)

(72) Inventor: Ryan Bruss, Sherwood, OR (US)

(21) Appl. No.: 19/420,405

(22) Filed: Dec. 15, 2025

Related U.S. Application Data
(60) Provisional application No. 63/742,933, filed on Jan. 8, 2025.

(51) Int. Cl. A61F 5/56

(52) U.S. Cl. CPC A61F 5/566

(57) ABSTRACT

An apparatus is provided that includes a top tray configured to conform to upper teeth, having a curved shape adapted to fit tooth and mouth contours and a recessed rear section with reduced thickness and length, terminating before posterior molars to minimize soft tissue contact. The apparatus comprises a bottom tray configured to conform to lower teeth, having a curved shape and asymmetrical inner ridge higher than the outer ridge to position the lower jaw forward. The apparatus comprises a top position adjuster attached to the top tray's underside, featuring a gap enabling vertical movement relative to a bottom position adjuster and an interlocking hook engaging with the top tray recess for detachable connection. The apparatus comprises the bottom position adjuster attached to the bottom tray's topside, featuring a protrusion within the gap limiting vertical movement and an interlocking hook engaging with the bottom tray recess for detachable connection.

### CROSS-REFERENCE TO RELATED APPLICATIONS

This application claims priority to U.S. Provisional Application No. 63/742,933, filed Jan. 8, 2025, which is hereby incorporated by reference in its entirety.

### FIELD OF INVENTION

The present disclosure relates to oral appliances for treating sleep-related breathing disorders, and more particularly to adjustable mandible positioning devices that maintain airway patency during sleep through controlled jaw positioning and vertical movement accommodation.

### BACKGROUND OF THE INVENTION

Obstructions or occlusions to an individual's airway can occur during sleep, often due to the relaxation of the palate or soft tissue surrounding the throat, which may restrict or completely block the flow of air. Additionally, the tongue may fall backward toward the throat while sleeping, further compromising the airway and adversely affecting the individual's ability to breathe. These obstructions can result in partial or complete blockage of the airway, leading to snoring, reduced or insufficient oxygen supply to the respiratory system, choking, interrupted sleep, and increased risks of serious health conditions such as heart disease and strokes.

Conventional treatment approaches for sleep-related breathing disorders include continuous positive airway pressure (CPAP) machines that deliver constant air pressure through a hose and mask system to maintain an open airway. However, CPAP machines can be cumbersome, noisy, and difficult to travel with, making them challenging for many individuals to use consistently. The masks and hoses may cause discomfort during sleep, and some individuals' bodies may not tolerate the positive airway pressure. Additionally, the cleaning and maintenance requirements for CPAP equipment can be burdensome, potentially leading to hygiene-related complications if neglected.

Surgical interventions represent another treatment option, involving the removal of tissue from the back of the mouth, such as the uvula or portions of the soft palate. However, surgical procedures carry inherent risks including post-surgical complications, poor healing, and infection. These invasive approaches may not be suitable or acceptable for all patients.

Oral appliances designed to address airway obstructions typically aim to stabilize or reposition the jaw and associated soft tissues to maintain an open and unobstructed airway. Many existing mandible adjustment devices rely on simple connection mechanisms or rigid structures that limit their adaptability and user comfort. These conventional designs often lack flexibility, causing discomfort during extended wear, and their rigid construction may fail to accommodate natural jaw movements, such as vertical compression or slight shifts in position. Additionally, the bulky design and excessive material in some models can lead to irritation in sensitive areas, such as the gums and tongue.

Current mandible adjustment apparatuses focus on stabilizing the lower jaw to keep the airway open. While these devices offer functional support, they present several challenges. Many designs do not account for individual anatomical variations or the natural mobility of the jaw, which leads to discomfort. Connection mechanisms often fail to provide a secure fit after molding, resulting in a lack of stability and performance degradation over time. Additionally, bulkiness and poorly placed materials may exacerbate irritation and make the apparatus unsuitable for long-term use.

### SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an apparatus is provided. The apparatus includes a top tray configured to be inserted into a mouth of a user. The top tray comprises an upper teeth channel. The upper teeth channel is configured to fit around or conform to two or more upper teeth of the user. A top position adjuster is coupled to the underside of the top tray. A bottom tray is configured to be inserted into the mouth of the user. The bottom tray comprises a lower teeth channel. The lower teeth channel is configured to fit around or conform to two or more lower teeth of the user. An inner ridge is present on a lingual side of the lower teeth channel. A height of the inner ridge is larger than a height of the outer ridge to promote a forward position of a jaw during use. A bottom position adjuster is coupled to the bottom tray and positioned to interface with the top position adjuster.

According to another aspect, an apparatus is provided. The apparatus includes a top tray configured to be inserted into a mouth of a user. The top tray comprises an upper teeth channel configured to fit around upper teeth of the user; and a first set of fastener sides on an underside of the top tray. The apparatus further includes a top position adjuster. The top position adjuster comprises a second set of fastener sides complimentary with the first set of fastener sides, wherein the top tray and the top position adjuster are joined by the first set of fastener sides and the second set of fastener sides to form an upper mandible tray; a set of position aligners on an underside of the top position adjuster. The set of position aligners comprises: a first row of aligners comprising: a first protrusion comprising a first peg, a first pin, or a first dowel; and a second protrusion comprising a second peg, a second pin, or a second dowel; and a second row of aligners comprising: a third protrusion comprising a third peg, a third pin, or a third dowel; and a fourth protrusion comprising a fourth post, a fourth peg, a fourth pin, or a fourth dowel. The top position adjuster further includes a recess on the underside of the top position adjuster. Only one lateral side of the recess is bounded by a wall on an outer edge of the top position adjuster. The apparatus further includes a bottom tray configured to be inserted into the mouth of the user. The bottom tray comprises a lower teeth channel configured to fit around lower teeth of the user; and a third set of fastener sides on a top side of the bottom tray. The apparatus further includes a bottom position adjuster, comprising: a fourth set of fastener sides complimentary with the third set of fastener sides, wherein the bottom tray and the bottom position adjuster are joined by the third set of fastener sides and the fourth set of fastener sides to form a lower mandible tray; a set of openings corresponding to the set of position aligners and positioned on a top side of the bottom position adjuster. The set of openings comprises a first cavity or a first channel configured to receive one of the first row of aligners or the second row of aligners; a second cavity or a second channel configured to receive one of the first row of aligners or the second row of aligners; and a third cavity or a third channel configured to receive one of the first row of aligners or the second row of aligners, wherein the set of position aligners and the set of openings are configured to connect to adjust an alignment of the upper mandible tray and the lower mandible tray laterally and longitudinally. The apparatus is configured so that as the user wears the upper mandible tray and the lower mandible tray, the set of position aligners is positioned to fit into the set of openings to connect the upper mandible tray and the lower mandible tray in a first position; the set of position aligners is positioned to fit into the set of openings to connect the upper mandible tray and the lower mandible tray in a second position that is different from the first position. The first position and the second position are laterally and longitudinally different. The upper mandible tray and the lower mandible tray are configured to prevent occlusion or obstruction of an airway of the user.

In another aspect, an apparatus is provided. The apparatus includes a top tray configured to be inserted into a mouth of a user. The top tray comprises an upper teeth channel including a plurality of crests and valleys in a wave pattern, configured to fit around upper teeth of the user and a first set of fastener sides on an underside of the top tray. The apparatus further includes a top position adjuster. The top position adjuster comprises a second set of fastener sides complimentary with the first set of fastener sides. The top tray and the top position adjuster are joined by the first set of fastener sides and the second set of fastener sides to form an upper mandible tray. The top position adjuster further comprises a set of position aligners on an underside of the top position adjuster. The set of position aligners comprises: a first row of aligners comprising a first peg and a second peg; and a second row of aligners comprising a third peg and a fourth peg. The top position adjuster further comprises a recess on the underside of the top position adjuster. Only one lateral side of the recess is bounded by a wall on an outer edge of the top position adjuster. The apparatus further includes a bottom tray configured to be inserted into the mouth of the user. The bottom tray comprises a lower teeth channel configured to fit around lower teeth of the user. An outer ridge is present on a labial side of the lower teeth channel. An inner ridge is present on a lingual side of the lower teeth channel. A height of the inner ridge is larger than a height of the outer ridge to promote a forward position of a jaw during use. A third set of fastener sides are on a top side of the bottom tray. The apparatus further includes a bottom position adjuster configured such that a front of the bottom position adjuster is positioned over a bottom incisor of the user as the user wears the lower mandible tray. The bottom position adjuster comprises a fourth set of fastener sides complimentary with the third set of fastener sides. The bottom tray and the bottom position adjuster are joined by the third set of fastener sides and the fourth set of fastener sides to form a lower mandible tray. The bottom position adjuster further comprises a set of openings corresponding to the set of position aligners and positioned on a top side of the bottom position adjuster. The set of openings comprises: a first channel configured to be aligned perpendicularly to a row of bottom teeth of the user and configured to receive one of the first row of aligners or the second row of aligners; a second channel configured to be aligned perpendicularly to a row of bottom teeth of the user and configured to receive one of the first row of aligners or the second row of aligners; and a third channel configured to be aligned perpendicularly to a row of bottom teeth of the user and configured to receive one of the first row of aligners or the second row of aligners. The set of position aligners and the set of openings are configured to connect to adjust an alignment of the upper mandible tray and the lower mandible tray laterally and longitudinally. The first channel, second channel and third channel are each curved towards the front of the bottom position adjuster or each comprise a ben. An outside of the bend faces towards the front of the bottom position adjuster. The apparatus is configured so that as the user wears the upper mandible tray and the lower mandible tray, the set of position aligners is positioned to fit into the set of openings to connect the upper mandible tray and the lower mandible tray in a first position. The set of position aligners is positioned to fit into the set of openings to connect the upper mandible tray and the lower mandible tray in a second position that is different from the first position. The first position and the second position are laterally and longitudinally different. The upper mandible tray and the lower mandible tray are configured to prevent occlusion or obstruction of an airway of the user.

The foregoing general description of the illustrative embodiments and the following detailed description thereof are merely exemplary aspects of the teachings of this disclosure and are not restrictive.

### BRIEF DESCRIPTION OF THE DRAWINGS

Non-limiting and non-exhaustive examples are described with reference to the following figures.

FIG. 1 illustrates an individual with a mandible adjustment apparatus in his/her mouth, according to an embodiment.

FIG. 2 is an isometric front view of a mandible adjustment apparatus with an upper mandible tray separated from a lower mandible tray, according to an embodiment.

FIG. 3A is a side view of a mandible adjustment apparatus in a first longitudinal position, according to an embodiment.

FIG. 3B is a side view of the mandible adjustment apparatus in a second longitudinal position, according to an embodiment.

FIG. 3C is a front view of the mandible adjustment apparatus of the subject disclosure, consistent with embodiments.

FIG. 4A is a bottom perspective exploded view of the mandible adjustment apparatus, according to an embodiment.

FIG. 4B is a top perspective exploded view of the mandible adjustment apparatus, according to an embodiment.

FIG. 5A is a top view of a top tray of a mandible adjustment apparatus, according to an embodiment.

FIG. 5B is a bottom view of a bottom tray of the mandible adjustment apparatus, according to an embodiment.

FIG. 6A is a top view of a bottom position adjuster in a first lateral position, according to an embodiment.

FIG. 6B is a top view of the bottom position adjuster in a second lateral position, according to an embodiment.

FIG. 7A is a top view of a bottom position adjuster with cavities for receiving protuberances from a top position adjuster, according to an embodiment.

FIG. 7B is a top view of a bottom position adjuster with the cavities within channels, according to an embodiment.

FIG. 7C is a top view of a bottom position adjuster with the channels curved backward, according to an embodiment.

FIG. 7D is a top view of a bottom position adjuster with the channels curved forward, according to an embodiment.

FIG. 8A is a rear, bottom perspective view of a top position adjuster, consistent with embodiments.

FIG. 8B is a bottom perspective view of the top position adjuster of FIG. 8A.

FIG. 9 is a bottom perspective view of the mandible adjustment apparatus of FIG. 2.

### DETAILED DESCRIPTION OF THE PREFERRED EMBODIMENTS

The following description sets forth exemplary aspects of the present disclosure. It should be recognized, however, that such description is not intended as a limitation on the scope of the present disclosure. Rather, the description also encompasses combinations and modifications to those exemplary aspects described herein.

Mandible adjustment apparatuses as disclosed herein will become better understood by reviewing the following detailed description in conjunction with the figures. The detailed description and figures provide merely examples of the various embodiments of mandible adjustment apparatuses. Many variations are contemplated for different applications and design considerations; however, for brevity and clarity, all the contemplated variations may not be individually described in the following detailed description. Those skilled in the art will understand how the disclosed examples may be varied, modified, and altered and not depart in substance from the scope of the examples described herein.

A conventional mandible adjustment apparatus may include a top and bottom tray designed to fit around a user's teeth, connected through a static mechanism to stabilize the jaw. Current apparatuses often rely on simple peg-and-slot connections or rigid structures that limit their adaptability and user comfort. These conventional designs typically lack flexibility, causing discomfort during extended wear, and their rigid construction may fail to accommodate natural jaw movements, such as vertical compression or slight shifts in position. Additionally, the bulky design and excessive material in some models can lead to irritation in sensitive areas, such as the gums and tongue.

Conventional mandible adjustment apparatuses focuses on stabilizing the lower jaw to keep the airway open. While these devices offer functional support, they present several challenges. Many designs do not account for individual anatomical variations or the natural mobility of the jaw, which leads to discomfort. Conventional connection mechanisms, such as straight pegs, often fail to provide a secure fit after molding, thereby resulting in a lack of stability and performance degradation over time. Additionally, bulkiness and poorly placed materials may exacerbate irritation and make the apparatus unsuitable for long-term use. These limitations highlight the need for an improved design that balances functionality, comfort, and adaptability.

Implementations of the disclosed mandible adjustment apparatus may address some or all of the problems described above. A mandible adjustment apparatus, as disclosed herein, may include top and bottom trays with a curved and ergonomic design, enhanced connection component, and a dual-material construction. One of the position adjusters uses a single wall configuration adjacent a recess that provide comfort while reducing material costs. The apparatus incorporates ridges with a wave pattern that has valleys and crests to reduce bulk while improving adaptability to individual oral anatomy. In addition, one of the ridges may have a larger profile than a ridge on the same mouthpiece platform. The height differential of the ridges on the same piece may promote forward positioning of the jaw during use. It may further include a strategically placed gap between the trays to allow for controlled vertical compression and movement for accommodating natural jaw mobility. The integration of hooks and an asymmetrical inner ridge on the bottom tray provides consistent forward positioning of the lower jaw for optimizing airway support and user comfort.

The disclosed mandible adjustment apparatuses will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations. However, for the sake of brevity and clarity, each and every contemplated variation is not individually described in the following detailed description.

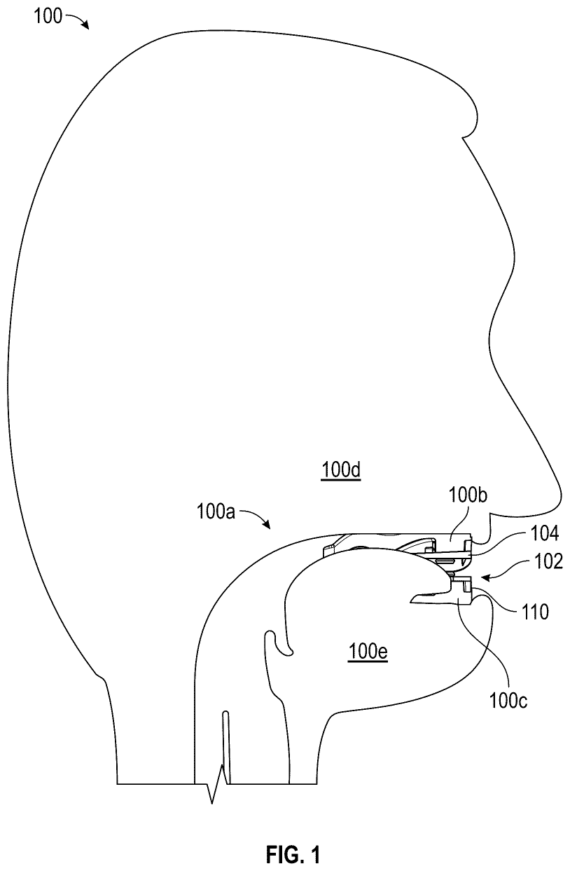

FIG. 1 illustrates an individual 100 with a mandible adjustment apparatus 102 in his/her mouth 100a, according to an embodiment. The mandible adjustment apparatus 102 may be configured to be inserted into the mouth 100a of the individual 100 to keep the individual's mouth 100a open and clear while the individual sleeps. The mandible adjustment apparatus 102 may include an upper mandible tray 104 and a lower mandible tray 110. The upper mandible tray 104 may be configured to fit around or conform to upper teeth 100b of the individual 100 and the lower mandible tray 110 may be configured to fit around or conform to lower teeth 100c of the individual 100 to separate the upper teeth 100b from the lower teeth 100c. The upper teeth 100b may include an upper bicuspid of the individual 100 and/or an upper molar of the individual 100. The lower teeth 100c may include a lower bicuspid of the individual 100 and/or a lower molar of the individual 100.

The mouth 100a of the individual 100 may be formed by an upper mandible 100d and a lower mandible 100e. During sleep, the lower mandible 100e my relax into a relaxed position which may cause obstruction of the individual's mouth 100a by, for example, a tongue of the individual pressing against a soft palate of the individual. The mandible adjustment apparatus 102 may maintain a position of the lower mandible 100e during sleep that prevents the lower mandible 100e from relaxing such that the mouth 100a becomes obstructed. Different individuals may have different relaxed positions of the lower mandible 100e. Over time, the relaxed position of the lower mandible 100e of the individual 100 may change. Accordingly, the mandible adjustment apparatus 102 may be adjusted to maintain the lower mandible 100e in different positions for different individuals or to maintain the lower mandible 100e in a different position for the individual 100 as the relaxed position of the lower mandible 100e changes.

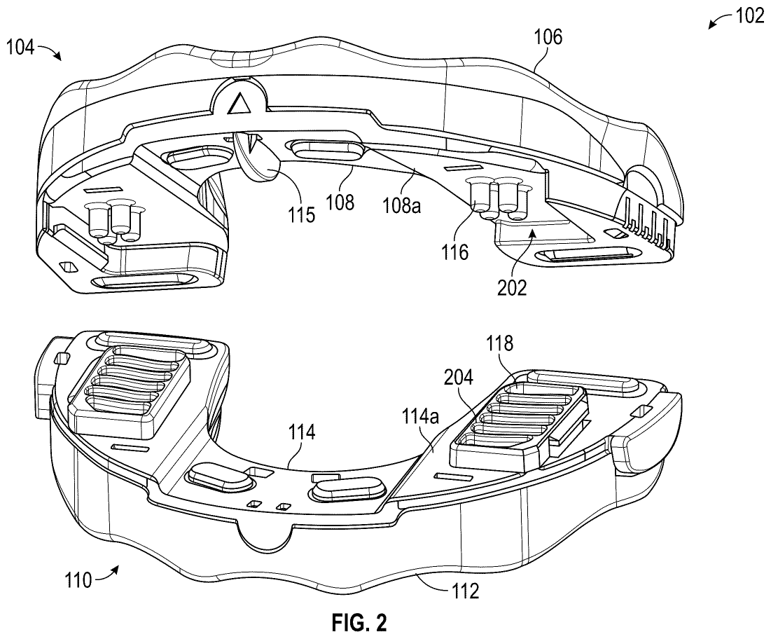

FIG. 2 illustrates the mandible adjustment apparatus 102 with the upper mandible tray 104 separated from the lower mandible tray 110, according to an embodiment. Some of the features in FIG. 2 are the same or similar to some of the features in FIG. 1 as noted by the same reference numbers, unless expressly described otherwise. Additionally, reference may be made to features shown in FIG. 1 and not shown in FIG. 2. In one embodiment, the upper mandible tray 104 may include a top tray 106 and a top position adjuster 108 that may be configured to connect together to form the upper mandible tray 104 that may be inserted around the upper teeth 100b of the individual 100. In one example, the top tray 106 may be a flexible cover with a top portion that includes a teeth channel that forms around the upper teeth of the individual to secure the upper mandible tray 104 to the upper teeth of the individual. In one example, the teeth channel of the top tray 106 may be a flexible rubber or plastic material that may be formed or molded to a shape corresponding to the upper teeth 100b of the individual 100. For example, the top tray 106 may be heated and pressed against the upper teeth 100b of the individual 100 such that the shape of the top tray 106 corresponds to the shape of the upper teeth 100b. The bottom portion of the top tray 106 may include fasteners to connect the bottom portion of the top tray 106 to a top portion of the top position adjuster 108. The fasteners may include pegs, pins, dowels, hooks, hangers, and so forth that may be inserted into openings in the top position adjuster 108 to connect the top tray 106 to the top position adjuster 108.

In another embodiment, the lower mandible tray 110 may include a bottom tray 112 and a bottom position adjuster 114 that may be configured to connect together to form the lower mandible tray 110 that may be inserted around the lower teeth 100c of the individual 100. In one example, the bottom tray 112 may be a flexible cover with a bottom portion that includes a teeth channel that forms around the lower teeth 100c of the individual 100 to secure the lower mandible tray 110 to the lower teeth of the individual. In one example, the teeth channel of the bottom tray 112 may be a flexible rubber or plastic material that may be formed or molded to a shape corresponding to the lower teeth 100c of the individual 100. For example, the bottom tray 112 may be heated and pressed against the lower teeth 100c of the individual 100 such that the shape of the bottom tray 112 conforms to the shape of the lower teeth 100c. The upper portion of the bottom tray 112 may include fasteners to connect the upper portion of the bottom tray 112 to a bottom portion of the bottom position adjuster 114. The fasteners may include pegs, pins, dowels, hooks, hangers, and so forth that may be inserted into cavities or openings in the bottom position adjuster 114 to connect the bottom tray 112 to the bottom position adjuster 114.

The top position adjuster 108 may be configured to connect with the bottom position adjuster 114 so that the mandible adjustment apparatus 102 is a single device or apparatus that may be inserted into the mouth 100a of the individual 100. A center stop 115 may be centralized on the bottom surface 108a of the top position adjuster 108 and sized to limit the downward action and pressure of the top position adjuster 108 on the bottom position adjuster 114. The center support 115 may have a curved, flat, bottom edge which adds comfort to some users (for example, by providing smoother edges that may irritate the tongue less). The center support 115 may be attached to either the bottom surface of the top position adjuster 108 or to the top surface 114a of the bottom position adjuster 114. In some embodiments, the center support 115 does not fully extend to the bottom position adjuster 114, leaving gap between the bottom surface of the center support 115 and the top surface 114a of the bottom position adjuster 114 (or vice versa in embodiments that project the center support 115 from the top of the bottom position adjuster 114). The gap allows for slight vertical cushioning movement between the top position adjuster 108 and the bottom position adjuster114. In one embodiment, the top position adjuster 108 may include one or more protuberances 116 that may extend downward from a bottom surface 108a of the top position adjuster 108. In one example, the bottom surface 108a of the top position adjuster 108 may include an indent or a recess 202 in which the protuberances 116 may be located. The recess 202 and/or the protuberances 116 may be positioned on the top position adjuster 108 to be directly under one or more of the upper teeth 100b of the individual 100 as the individual 100 wears the upper mandible tray 104. The bottom position adjuster 114 may include one or more cavities or channels 118 that may be located along the top surface 114a of the bottom position adjuster 114. In another example, the cavities or channels 118 may be formed in a raised portion 204 of the bottom position adjuster 114 that extends upward from the top surface 114a of the bottom position adjuster 114. The channels 118 may be positioned on the bottom position adjuster 114 to be directly over one or more of the lower teeth 100c of the individual 100 as the individual 100 wears the lower mandible tray 110. The channels 118 may be aligned perpendicular to a row of the lower teeth 100c of the individual 100. For example, the channels 118 may be aligned perpendicular to a row of molars of the individual 100.

The protuberances 116 may be male-type fasteners that are configured to mate with the channels 118. The channels 118 may be female-type fasteners. For example, the protuberances 116 and the channels 118 may be located on corresponding surfaces of the top position adjuster 108 and the bottom position adjuster 114 to connect the top position adjuster 108 with the bottom position adjuster 114. The protuberances 116 may be inserted into one or more of the channels 118. In one embodiment, the bottom position adjuster 114 may include multiple channels 118 that may be configured to receive the protuberances 116. The multiple channels 118 may allow the individual 100 to adjust the upper mandible tray 104 relative to the lower mandible tray 110 by inserting the protuberances 116 into different channels 118 or within different portions of the same channels 118. In one example, the upper mandible tray 104 may be adjusted longitudinally (i.e. backward and forward) relative to the lower mandible tray 110 by inserting the protuberances 116 into different channels 118. In another example, the upper mandible tray 104 may be adjusted laterally (i.e. right to left) relative to the lower mandible tray 110 by inserting the protuberances 116 different portions of the same channels 118.

The adjustability of the upper mandible tray 104 relative to the lower mandible tray 110 may allow a user to position each of the upper mandible tray 104 and the lower mandible tray 110 at optimal positions such that the upper mandible tray 104 and the lower mandible tray 110 are comfortable when the mandible adjustment apparatus 102 is positioned within the mouth 100a of the individual 100. For example, when the upper teeth 100b of the individual 100 extend farther forward than the lower teeth 100c, the mandible adjustment apparatus 102 may be configured to fit the mouth 100a of the individual 100. In another example, when the upper teeth 100b of the individual 100 extend farther to the left or right of the lower teeth 100c, the mandible adjustment apparatus 102 may be configured to fit the mouth 100a of the individual 100.

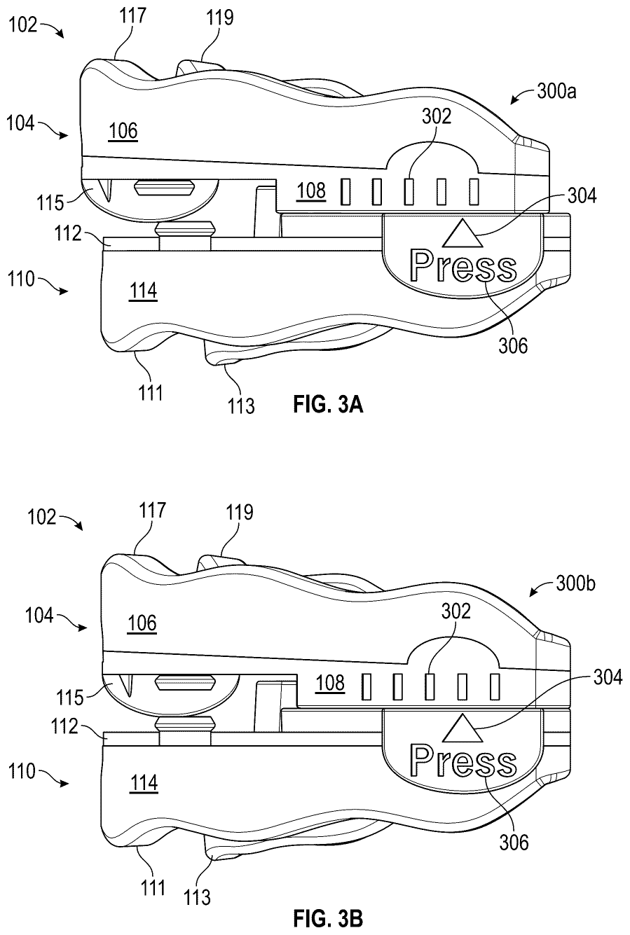

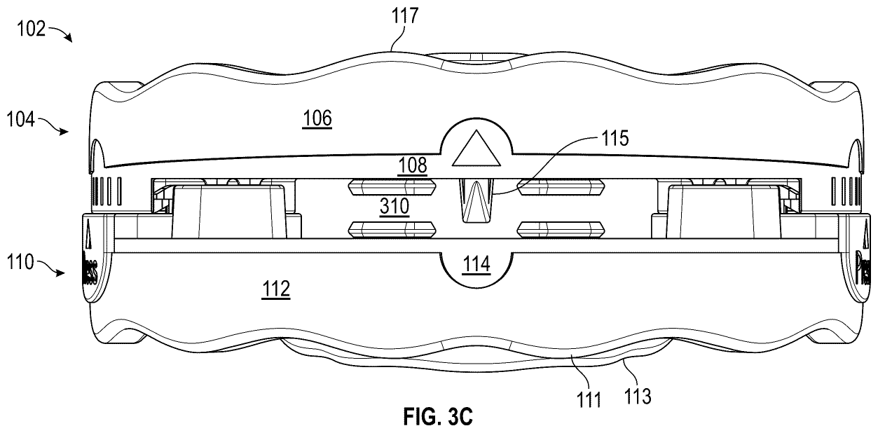

FIG. 3A illustrates a side view of the mandible adjustment apparatus 102 in a first longitudinal position 300a, according to an embodiment. Some of the features in FIG. 3A are the same or similar to some of the features in FIGS. 1-2 as noted by the same reference numbers, unless expressly described otherwise. Additionally, reference may be made to features shown in any of FIGS. 1-2 and not shown in FIG. 3A. The upper mandible tray 104 may include a set of alignment marks 302. The lower mandible tray 110 may include an alignment indicator 304. The lower mandible tray 110 may also include an orientation indicator 306. A position of the alignment indicator 304 relative to the alignment marks 302 may indicate a configuration of a bite of the individual 100. The alignment marks 302 may indicate an alignment interval of the upper mandible tray 104 relative to the lower mandible tray 110. For example, the individual may have a horizontal overbite. Alignment of the alignment indicator 304 with a rearward alignment mark may indicate the individual has the horizontal overbite. The relative position of the alignment indicator 304 relative to the alignment marks 302, i.e. the alignment interval, may indicate a degree of the overbite.

In one embodiment, the alignment marks 302 and the alignment indicator 304 may allow the individual 100 to reassemble the mandible adjustment apparatus 102 to an optimal alignment for the individual 100. For example, the mandible adjustment apparatus 102 may be disassembled to be cleaned. The upper mandible tray 104 may be separated from the lower mandible tray 110. The top tray 106 may be separated from the top position adjuster 108. The bottom tray 112 may be separated from the bottom position adjuster 114. The separated pieces of the mandible adjustment apparatus 102 may be cleaned and then reassembled. The individual 100 may use the alignment marks 302 and the alignment indicator 304 to align the upper mandible tray 104 with the lower mandible tray 110 as the upper mandible tray 104 and the lower mandible tray 110 are reconnected.

In various embodiments, the alignment marks 302 may be on the upper mandible tray 104 and/or the lower mandible tray 110. The alignment marks 302 may be on the top tray 106 and/or the top position adjuster 108. The alignment marks 302 may be on the bottom tray 112 and/or on the bottom position adjuster 114. The alignment indicator 304 may be on the upper mandible tray 104 and/or the lower mandible tray 110. The alignment indicator 304 may be on the top tray 106 and/or the top position adjuster 108. The alignment indicator 304 may be on the bottom tray 112 and/or the bottom position adjuster 114. In various embodiments, the mandible adjustment apparatus 102 may include corresponding alignment marks 302 on the upper mandible tray 104 and on the lower mandible tray 110. In various embodiments, the mandible adjustment apparatus 102 may include corresponding alignment indicators 304 on the upper mandible tray 104 and on the lower mandible tray 110.

The orientation indicator 306 may indicate to the individual a correct orientation of the mandible adjustment apparatus 102 for inserting into the mouth 100a of the individual 100. The orientation indicator 306 may indicate to the individual 100 the correct orientation by placement on one of the mandible trays. For example, placement of the orientation indicator 306 on the lower mandible tray 110 may indicate to the individual 100 which of the mandible trays is the lower mandible tray 110, and that the mandible tray with the orientation indicator 306 corresponds to the lower teeth 100c of the individual 100. Placement of the orientation indicator 306 on the upper mandible tray 104 may indicate to the individual that the mandible tray with the orientation indicator 306 corresponds to the upper teeth 100b of the individual 100. In one embodiment, the orientation indicator 306 may include one or more letters and/or words. The orientation of the letters and/or words may indicate to the individual 100 a correct orientation of the mandible adjustment apparatus 102 in the mouth 100a of the individual 100.

FIG. 3B illustrates a side view of the mandible adjustment apparatus 102 in a second longitudinal position 300b, according to an embodiment. Some of the features in FIG. 3B are the same or similar to some of the features in FIGS. 1-3A as noted by the same reference numbers, unless expressly described otherwise. Additionally, reference may be made to features shown in any of FIGS. 1-3A and not shown in FIG. 3B. As indicated by a relative position of the alignment indicator 304 relative to the alignment marks 302, the second longitudinal position 300b is different from the first longitudinal position 300a. The first longitudinal position 300a in FIG. 3A may correspond to the individual 100 having an overbite. The second longitudinal position 300b may correspond to the individual 100 having a normal bite. Accordingly, the mandible adjustment apparatus 102 may allow the individual 100 to track the progress of treatment for an overbite or an underbite.

FIG. 3C illustrates a head-on view of the mandible adjustment apparatus 102, according to an embodiment. Some of the features in FIG. 3C are the same or similar to some of the features in FIGS. 1-3B as noted by the same reference numbers, unless expressly described otherwise. Additionally, reference may be made to features shown in any of FIGS. 1-3B and not shown in FIG. 3C. The mandible adjustment apparatus 102 may include a spacer tab 308. The spacer tab 308 may be positioned between incisors of the individual 100 as the mandible adjustment apparatus 102 is worn by the individual 100 in the mouth 100a of the individual 100. The spacer tab 308 may maintain an open airway 310 between the upper mandible tray 104 and the lower mandible tray 110. The spacer tab 308 may prevent the individual 100 from closing the airway 310, such as by biting and/or grinding the individual's teeth during sleep. The spacer tab 308 may, in an embodiment, be integrated with and extend from the top position adjuster 108. The spacer tab 308 may, in another embodiment, be integrated with and extend from the bottom position adjuster 114. The spacer tab 308 may be aerodynamic, such as by having a rounded surface, to minimize drag and turbulence within the mouth 100a of the individual 100 as the individual 100 breathes. This may enhance a comfort of the mandible adjustment apparatus 102 relative to other embodiments where the spacer tab 308 is not aerodynamically-shaped.

In another embodiment, the airway 310 may extend from a front of the mandible adjustment apparatus 102 to a back of the mandible adjustment apparatus 102. A center portion of the top tray 106 and the top position adjuster 108 may curve or extend upward relative to the bottom tray 112 and the bottom position adjuster 114 to provide the airway 310. In another example, a center portion of the bottom tray 112 and the bottom position adjuster 114 may curve or extend downward relative to the top tray 106 and the top position adjuster 108 to provide the airway 310. In another example, a center portion of the top tray 106 and the top position adjuster 108 may curve or extend upward relative to the bottom tray 112 and the bottom position adjuster 114 and a center portion of the bottom tray 112 and the bottom position adjuster 114 may curve or extend downward relative to the top tray 106 and the top position adjuster 108 to provide the airway 310. The airway 310 may be a cutout pattern in the center of the mandible adjustment apparatus 102 to provide a secure fit in the mouth of the user while providing a relatively large breathing space for the user.

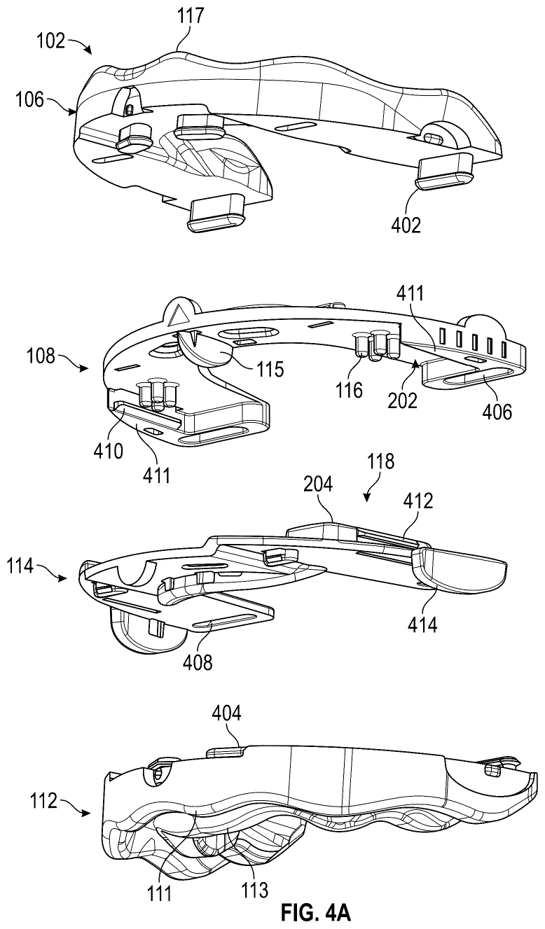

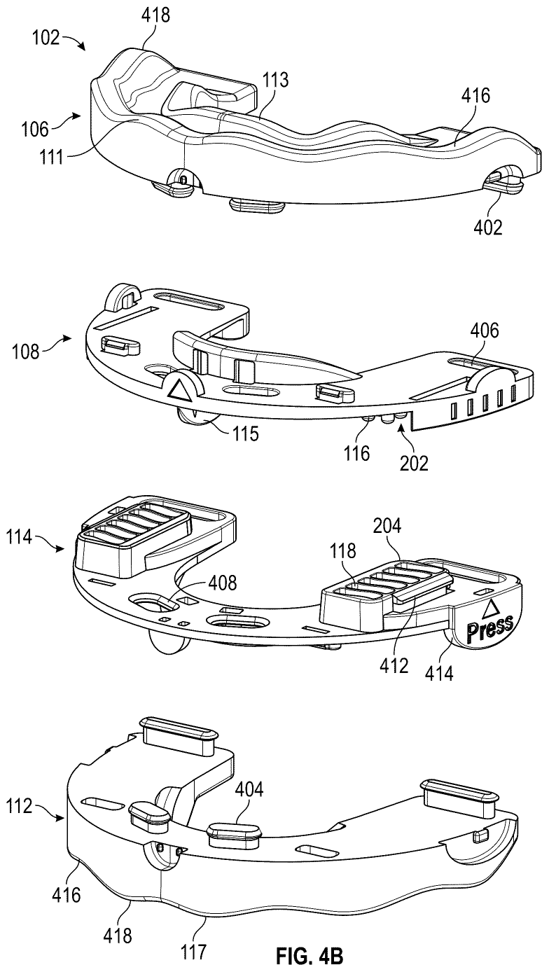

FIG. 4A illustrates a bottom perspective exploded view of the mandible adjustment apparatus 102, according to an embodiment. Some of the features in FIG. 4A are the same or similar to some of the features in FIGS. 1-3C as noted by the same reference numbers, unless expressly described otherwise. Additionally, reference may be made to features shown in any of FIGS. 1-3C and not shown in FIG. 4A. As discussed above, the mandible adjustment apparatus 102 may include the top tray 106, the top position adjuster 108, the bottom tray 112, and the bottom position adjuster 114. A first set of male fasteners 402 may extend from the top tray 106. A second set of male fasteners 404 may extend from the bottom tray 112. A first set of female fasteners 406 may be formed in the top position adjuster 108. A second set of female fasteners 408 may be formed in the bottom position adjuster 114.

The first set of male fasteners 402 may insert into the first set of female fasteners 406 to join the top tray 106 to the top position adjuster 108. The first set of fastener sides, i.e. the first set of male fasteners 402 and the first set of female fasteners 406, may include a hook-and-catch snap joint. The second set of male fasteners 404 may insert into the second set of female fasteners 408 to join the bottom tray 112 to the bottom position adjuster 114. The second set of fastener sides, i.e. the second set of male fasteners 404 and the second set of female fasteners 408, may include a hook-and-catch snap joint. In an embodiment, the female fasteners may include openings and the male fasteners may include protrusions with hooks or catches. The male fasteners may be compressible to press through the openings, and the hooks or catches may catch against edges of the opening to prevent the male fasteners from pulling through the openings The female fasteners may be stretchable to stretch as the male fasteners press through the openings. In one embodiment, the first set of male fasteners 402 may extend from the top position adjuster 108 and the first set of female fasteners 406 may be formed in the top tray 106. In another embodiment, the second set of male fasteners 404 may extend from the bottom position adjuster 114 and the second set of female fasteners 408 may be formed in the bottom tray 112.

The top position adjuster 108 may include the recess 202 in which the protuberances 116 may be disposed. The recess 202 may include a catch 410. The bottom position adjuster 114 may include the raised portion 204 in which the channels 118 may be disposed. The raised portion 204 may include a hook 412. Together, the recess 202 and the raised portion 204 may form a snap joint. As the upper mandible tray 104 is brought together with the lower mandible tray 110, the raised portion 204 may insert into the recess 202. As the raised portion 204 inserts into the recess 202, the protuberances 116 may insert into the channels 118. The hook 412 may snap over the catch 410, securing the upper mandible tray 104 to the lower mandible tray 110. The bottom position adjuster 114 may include a pressing surface 414. In an embodiment, the bottom position adjuster 114 may include two pressing surfaces 414 positioned opposite each other on the bottom position adjuster 114. Pressure on the pressing surfaces 414 may deflect the hook 412 away from the catch 410 and release the raised portion 204 from the recess 202. Accordingly, pressure on the pressing surface 414 may release the upper mandible tray 104 from the lower mandible tray 110.

In various embodiments, the elements described above may be arranged differently. For example, the raised portion 204 may be on the top position adjuster 108 and the recess 202 may be formed in the bottom position adjuster 114. The pressing surface 414 may be formed on the top position adjuster 108. The hook 412 may be disposed on the protuberances 116 and the catch 410 may be disposed in the channels 118. Accordingly, the protuberances 116 and the channels 118 may form a hook-and-catch snap joint.

The top tray 106, the top position adjuster 108, the bottom tray 112, and/or the bottom position adjuster 114 may be formed of a plastic material such as a thermoplastic, an injection-molded plastic, and so forth. The top tray 106 may be of unibody construction, and/or the bottom tray 112 may be of unibody construction. The top position adjuster 108 may be of unibody construction, and/or the bottom position adjuster 114 may be of unibody construction. The top tray 106 and the bottom tray 112 may be formed of the same material, and the top position adjuster 108 and the bottom position adjuster 114 may be formed of the same material as each other but a different material than the top tray 106 and the bottom tray 112. The top tray 106 and/or the bottom tray 112 may be configured to fit around or conform to two or more of the upper teeth 100b and/or the lower teeth 100c, respectively. The top tray 106, the top position adjuster 108, the bottom tray 112, and/or the bottom position adjuster 114 may be horseshoe-shaped. Accordingly, the top position adjuster 108 may extend around the mouth 100a of the individual 100 from top right-side molars to top left-side molars of the individual 100. Similarly, the bottom position adjuster 114 may extend around the mouth 100a of the individual 100 from bottom right-side molars to bottom left-side molars of the individual 100. The top position adjuster 108 may include a right-side set of protuberances 116 and a left-side set of protuberances 116. The bottom position adjuster 114 may include a right-side set of channels 118 and a left-side set of channels 118.

FIG. 4B illustrates a top perspective exploded view of the mandible adjustment apparatus 102, according to an embodiment. Some of the features in FIG. 4B are the same or similar to some of the features in FIGS. 1-4A as noted by the same reference numbers, unless expressly described otherwise. Additionally, reference may be made to features shown in any of FIGS. 1-4A and not shown in FIG. 4B. From the top perspective, the channels 118 in the raised portion 204 are visible.

In one embodiment, the top tray 106 and/or the bottom tray 112 may have a wave pattern. The wave pattern may enable the top tray 106 and/or the bottom tray 112 to better fit around the upper teeth 100b and gums or lower teeth 100c and gums, respectively, of the individual 100 by reducing the overall volume of the mandible adjustment apparatus 102. For example, valleys 416 or bottom portions of the wave pattern of the top tray 106 and/or the bottom tray 112 may provide an area of space for the gums of the individual to spread out to or breathe while the crests 418 or top portions of the wave pattern may secure the top tray 106 and/or the bottom tray 112 to the teeth of the individual. The number of valleys 416 and crests 418 in the wave patterns are not intended to be limiting and may be increased or decreased based on the size of the user's mouth, the sensitivity of the user's teeth or gums, and so forth.

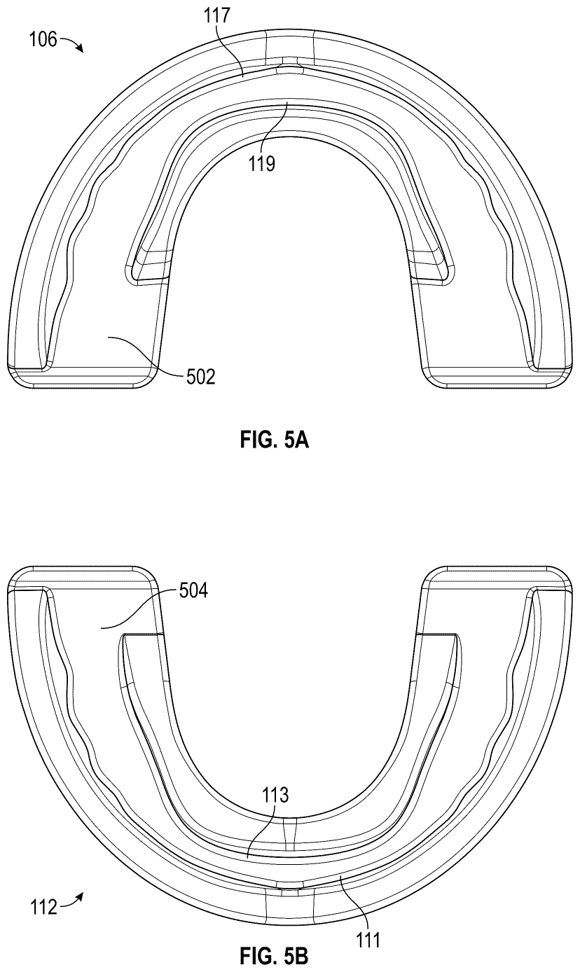

FIG. 5A illustrates a top view of the top tray 106, according to an embodiment. Some of the features in FIG. 5A are the same or similar to some of the features in FIGS. 1-4B as noted by the same reference numbers, unless expressly described otherwise. Additionally, reference may be made to features shown in any of FIGS. 1-4B and not shown in FIG. 5A. The top tray 106 may include an upper teeth channel 502. The upper teeth channel 502 may be configured to receive one or more upper teeth of the individual, such as the upper teeth 100b.

The top tray 106 may be formed of a remoldable polymer that softens when heated above a temperature, such as above 110° F. In an embodiment, the remoldable polymer may retain shape at temperatures up to 110° F., 120° F., 130° F., or 140° F. In an embodiment, the remoldable polymer may soften at temperatures between 110° F. and 200° F., between 120° F. and 200° F., between 130° F. and 200° F., or between 140° F. and 200° F. The top tray 106 may be remoldable to allow the upper teeth channel 502 to conform around the upper teeth 100b of the individual 100. For example, the top tray 106 may be heated to soften the top tray 106, and the individual 100 may insert the top tray in the individual's mouth 100a around the upper teeth 100b of the individual. The upper teeth channel 502 may conform to a shape of the upper teeth 100b. As the top tray 106 cools, the upper teeth channel 502 may retain the shape of the upper teeth 100b.

The top position adjuster 108 may be formed of a rigid polymer having a higher hardness and/or thermal resistance than the top tray 106. For example, whereas the top tray 106 may soften at temperatures ranging from 110° F. to 200° F., the top position adjuster 108 may remain rigid at temperatures ranging from 110° F. to 200° F. In an embodiment, the remoldable polymer may include ethylene-vinyl acetate. In an embodiment, the rigid polymer may be formed of polyurethane plastic. The top tray 106 may be softer than the top position adjuster to form a compressible barrier between the upper teeth 100b and the top position adjuster 108. The top position adjuster 108 may be rigid to hold open the mouth 100a of the individual 100 and to affix the upper mandible tray 104 to the lower mandible tray 110.

FIG. 5B illustrates a bottom view of the bottom tray 112, according to an embodiment. Some of the features in FIG. 5B are the same or similar to some of the features in FIGS. 1-5A as noted by the same reference numbers, unless expressly described otherwise. Additionally, reference may be made to features shown in any of FIGS. 1-5A and not shown in FIG. 5B. The bottom tray 112 may include a lower teeth channel 504. The lower teeth channel 504 may be configured to receive one or more lower teeth of the individual 100, such as the lower teeth 100c.

The bottom tray 112 may be formed of the remoldable polymer that softens when heated. In an embodiment, the remoldable polymer may retain shape at temperatures up to 110° F., 120° F., 130° F., or 140° F. In an embodiment, the remoldable polymer may soften at temperatures between 110° F. and 200° F., between 120° F. and 200° F., between 130° F. and 200° F., or between 140° F. and 200° F. The bottom tray 112 may be remoldable to allow the lower teeth channel 504 to conform around the lower teeth 100c of the individual 100. For example, the bottom tray 112 may be heated to soften the bottom tray 112, and the individual 100 may insert the top tray in the individual's mouth 100a around the lower teeth 100c of the individual 100. The lower teeth channel 504 may conform to a shape of the lower teeth 100c. As the bottom tray 112 cools, the lower teeth channel 504 may retain the shape of the lower teeth 100c.

The bottom position adjuster 114 may be formed of a rigid polymer having a higher hardness and thermal resistance than the bottom tray 112. For example, whereas the bottom tray 112 may soften at temperatures ranging from 110° F. to 200° F., the bottom position adjuster 114 may remain rigid at temperatures ranging from 110° F. to 200° F. In an embodiment, the remoldable polymer may include ethylene-vinyl acetate. In an embodiment, the rigid polymer may be formed of polyurethane plastic. The bottom tray 112 may be softer than the bottom position adjuster 114 to form a compressible barrier between the lower teeth 100c and the bottom position adjuster 114. The bottom position adjuster 114 may be rigid to hold open the mouth 100a of the individual 100 and to affix the lower mandible tray 110 to the upper mandible tray 104.

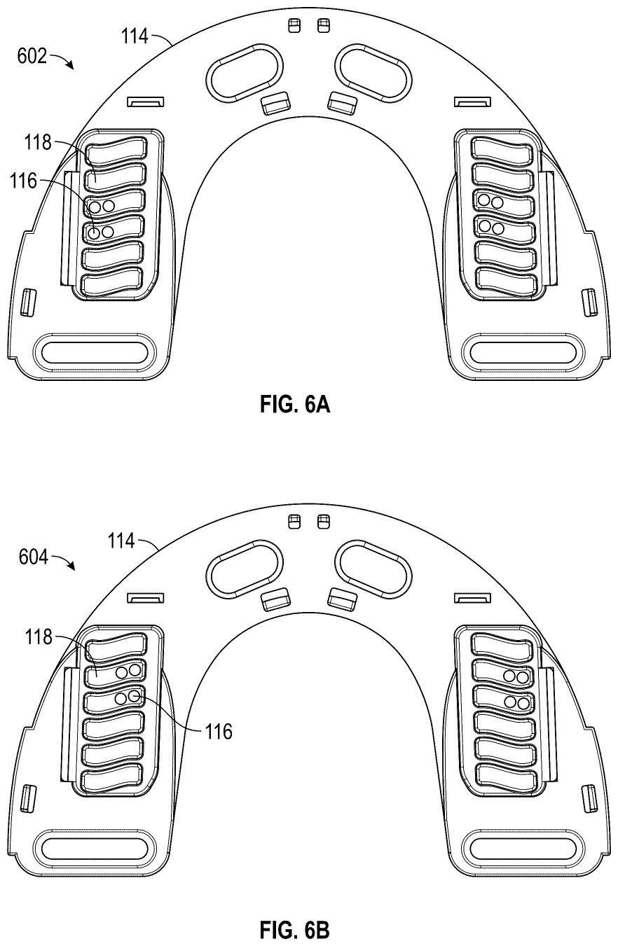

FIG. 6A illustrates a top view of the bottom position adjuster 114 in a first lateral position 602, according to an embodiment. Some of the features in FIG. 6A are the same or similar to some of the features in FIGS. 1-5B as noted by the same reference numbers, unless expressly described otherwise. Additionally, reference may be made to features shown in any of FIGS. 1-5B and not shown in FIG. 6A. The first lateral position 602 of the top position adjuster 108 relative to the bottom position adjuster 114 is reflected in the relative position of the protuberances 116 in the channels 118. The top position adjuster 108 may be shifted left relative to the bottom position adjuster 114. The protuberances 116 may shift in the channels 118, which may allow the lower mandible 100e of the individual 100 to shift side to side. The protuberances 116 may hook into the channels 118 to secure the top position adjuster 108 to the bottom position adjuster 114.

FIG. 6B illustrates a top view of the bottom position adjuster 114 in a second lateral position 604, according to an embodiment. Some of the features in FIG. 6B are the same or similar to some of the features in FIGS. 1-6A as noted by the same reference numbers, unless expressly described otherwise. Additionally, reference may be made to features shown in any of FIGS. 1-6A and not shown in FIG. 6B. The second lateral position 604 of the top position adjuster 108 relative to the bottom position adjuster 114 may be reflected in the relative position of the protuberances 116 in the channels 118. The top position adjuster 108 may be shifted right relative to the bottom position adjuster 114. The top position adjuster 108 may also be shifted forward relative to the bottom position adjuster 114, reflecting an overbite of the individual 100. The protuberances 116 may shift in the channels 118, which may allow the lower mandible 100e of the individual 100 to shift side to side. However, the protuberances 116 and channels 118 may prevent the lower mandible 100e of the individual 100 from sliding backward or forward relative to the upper mandible 100d. The protuberances 116 may hook into the channels 118 to secure the top position adjuster 108 to the bottom position adjuster 114.

The protuberances 116 and channels 118 may also prevent the top position adjuster 108 from unintentionally coming apart from the bottom position adjuster 114. In another embodiment, the protuberances 116 may be slightly misaligned with the channels 118 and/or openings in the channels 118. By pushing in on the pressing surface 414 of the upper mandible tray 104 and/or the lower mandible tray 110, the user may adjust the position or configuration of the top position adjuster 108 and/or the bottom position adjuster 114. The channels 118 may have a wavy shape. The protuberances 116 may be flexible and may bend to conform to the wavy shape of the channels 118.

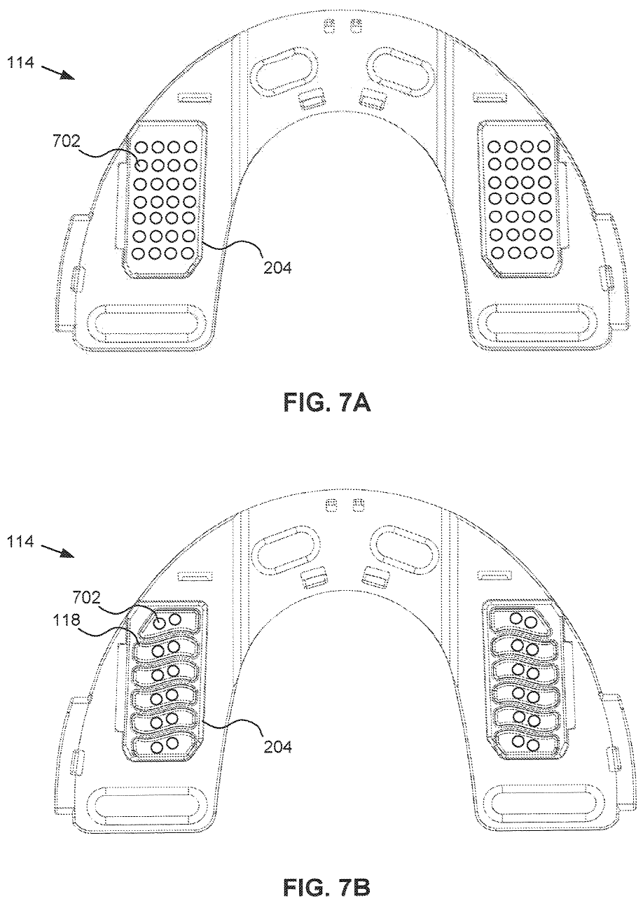

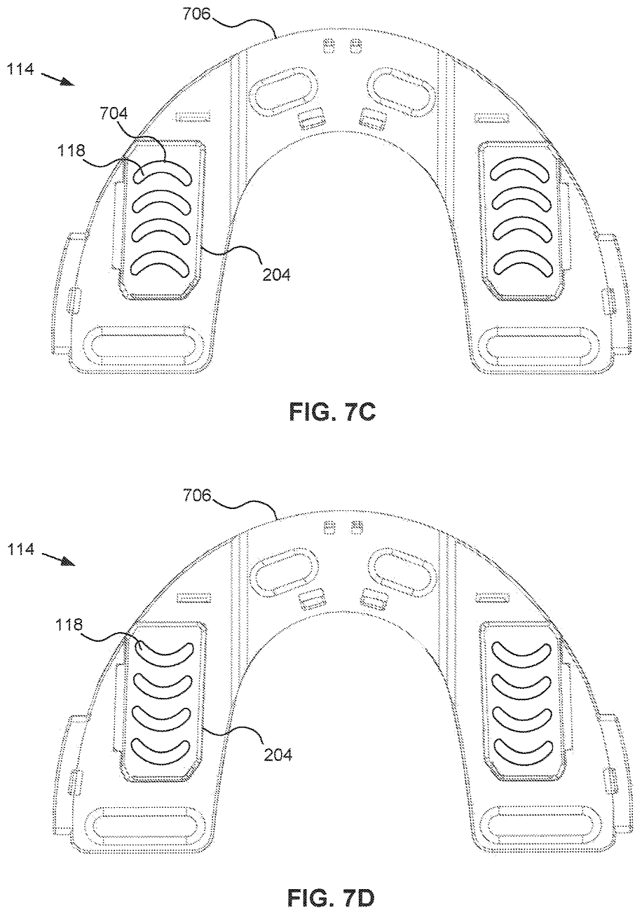

FIG. 7A illustrates the bottom position adjuster 114 with cavities 702 for receiving the protuberances 116 from the top position adjuster 108, according to an embodiment. Some of the features in FIG. 7A are the same or similar to some of the features in FIGS. 1-6B as noted by the same reference numbers, unless expressly described otherwise. Additionally, reference may be made to features shown in any of FIGS. 1-6B and not shown in FIG. 7A. In an embodiment, the raised portion 204 of the bottom position adjuster 114 may include the cavities 702. The cavities 702 may receive the protuberances 116 to secure the top position adjuster 108 in place relative to the bottom position adjuster 114.

FIG. 7B illustrates the bottom position adjuster 114 with the cavities 702 within the channels 118, according to an embodiment. Some of the features in FIG. 7B are the same or similar to some of the features in FIGS. 1-7A as noted by the same reference numbers, unless expressly described otherwise. Additionally, reference may be made to features shown in any of FIGS. 1-7A and not shown in FIG. 7B. In one embodiment, the channels 118 may include the cavities 702 to receive the protuberances 116 of the top position adjuster 108. The channels 118 may extend a partial depth in the raised portion 204, and the cavities 702 may extend to a further depth in the raised portion 204 beyond the depth of the channels 118. The channels 118 may include sloped sides that slope towards the cavities 702. The sloped sides may guide the protuberances 116 towards the cavities 702.

In an embodiment, a channel 118 may include 2 cavities 702. In another embodiment, a channel 118 may include 4 cavities 702. As discussed above, the multiple channels 118 and cavities 702 may allow a user to adjust the upper mandible tray 104 relative to the lower mandible tray 110 by inserting the protuberances 116 into different channels 118 or within different cavities 702 of the same channels 118.

FIG. 7C illustrates the bottom position adjuster 114 with the channels 118 curved backward, according to an embodiment. Some of the features in FIG. 7C are the same or similar to some of the features in FIGS. 1-7B as noted by the same reference numbers, unless expressly described otherwise. Additionally, reference may be made to features shown in any of FIGS. 1-7B and not shown in FIG. 7C. In an embodiment, the channel 118 may include a bend. An outside 704 of the bend may face towards a front end 706 of the bottom position adjuster 114. The front end 706 of the bottom position adjuster may be positioned over a bottom incisor of the individual 100 as the individual 100 wears the lower mandible tray 110.

FIG. 7D illustrates the bottom position adjuster 114 with the channels 118 curved forwards, according to an embodiment. Some of the features in FIG. 7D are the same or similar to some of the features in FIGS. 1-7C as noted by the same reference numbers, unless expressly described otherwise. Additionally, reference may be made to features shown in any of FIGS. 1-7C and not shown in FIG. 7D. In an embodiment, the channel 118 may be curved towards the front end 706 of the bottom position adjuster 114.

### Single Wall Top Position Adjuster

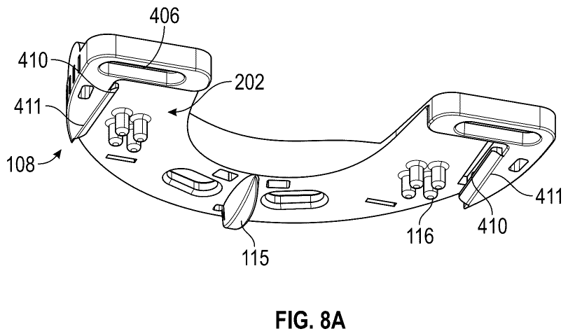

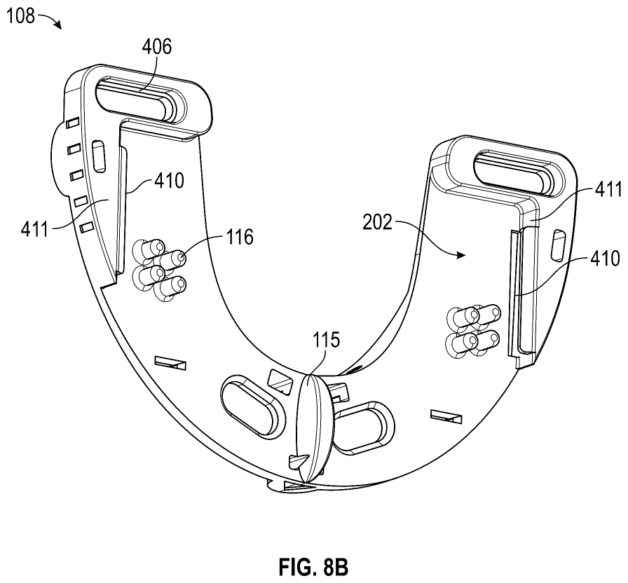

Referring now to FIG. 8A and FIG. 8B, the top position adjuster 108 is shown in further detail. From this perspective it can be seen that in one embodiment of the top position adjuster 108, the recess 202 is bounded only on one of its lateral sides, (the outer edge) by a single wall 411 that supports the catch 410. The recess 202 may also be bounded at its rear side by the female fastener 406. The interior facing side of the recess 202 is open or unbounded by any obstruction, as is the front side (in front of the protuberances 116). The single wall configuration may provide advantages over other configurations. For example, the open interior of the recess 202 may be more comfortable for some users on the lingual side since the sides of the tongue do not have to engage any element such as a wall which is present in some other devices. This provides more room and comfort especially for those with larger tongues. In addition, the single wall 411 configuration may provide unexpectedly competitive performance results at a lower cost of manufacturing by reducing materials used in the construction of the device while still providing the primary function of adjusting the mandible.

### Mandible Forward Adjustment

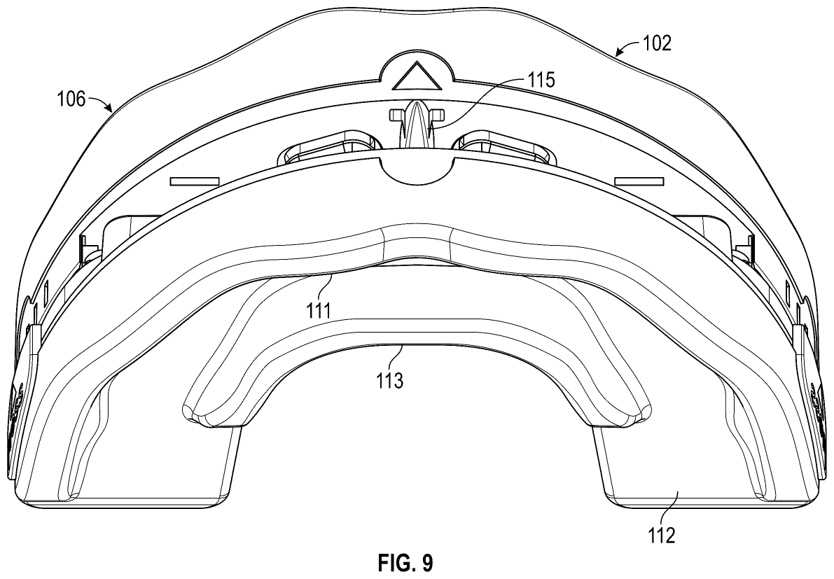

Referring back to FIG. 3A, FIG. 3B, FIG. 3C, FIG. 4A, FIG. 4B, FIG. 5A, FIG. 5B and now to FIG. 9, the mandible adjustment 102 apparatus may incorporate forward jaw positioning through the coordinated operation of the position adjuster 108 and the structural characteristics of the bottom tray 112 that work together to maintain the lower jaw in a forward position relative to the upper jaw. The top tray 102 may include an outer ridge 117 on the labial side and an inner ridge 119 on the lingual side. The outer ridge 117 and the inner ridge 119 may have heights that are roughly equal as can be seen in FIG. 3A, FIG. 3B, and FIG. 3C.

In one embodiment, the bottom tray 112 may include an inner ridge 113 that extends along the lingual side of the bottom tray 112 that provides enhanced engagement with the lower teeth to support forward jaw positioning. The inner ridge 113 may be recessed from the rear edge of the bottom tray 112. The inner ridge 113 functions by redistributing the contact forces of the tray across the anterior and mid-sections of the upper teeth. In this regard, no or limited stress is applied to the posterior region of the oral cavity. This relief minimizes interference with the tongue's natural movement, allowing for unobstructed airflow, which maintains an open airway during use. Additionally, this recessed configuration helps reduce the bulk of the apparatus at the posterior end and makes the apparatus less intrusive and more comfortable for the user. The inner ridge 113 may be asymmetrical to an outer ridge 111 of the bottom tray 112. For example, the height of the inner ridge 113 may be positioned to extend farther away from the lower teeth channel 504 than outer ridge 111 that extends along the labial side of the bottom tray 112, creating a differential height configuration that enhances retention and positioning capabilities. The difference in height can be seen in in FIG. 3A, FIG. 3B, FIG. 3C, and FIG. 9. The height differential between the inner ridge 112 and the outer ridge 111 may create a structural bias that encourages forward positioning of the lower jaw while providing secure engagement with the dental structures that anchor the bottom tray within the mouth.

The asymmetrical ridge design may provide enhanced retention capabilities by creating multiple contact zones with the lower teeth that distribute positioning forces across a larger surface area. The inner ridge 113 may engage with the lingual surfaces of the lower teeth, providing primary retention and positioning control that maintains the forward jaw position throughout extended use periods. The outer ridge 111 may engage with the labial surfaces of the lower teeth, providing secondary retention that complements the primary positioning function of the inner ridge while maintaining overall stability of the bottom tray. The height differential between the ridges 111 and 113 may create a wedging effect that locks the bottom tray 112 against the dental structures in a manner that resists backward movement of the lower jaw while allowing for natural vertical movements that occur during jaw compression and expansion cycles.

As may be further appreciated, the integration of the position adjusters (108; 114) with the asymmetrical ridge design may create a comprehensive jaw support system that maintains consistent forward positioning while accommodating individual anatomical variations and movement patterns. The protuberances 116 and channels 118 within the position adjusters (108; 114) may allow for customization of the apparatus to accommodate different jaw relationships and bite patterns, while the asymmetrical ridge design ensures that the lower jaw remains in the forward position needed for effective airway support regardless of the macro-level adjustments made through the position adjuster system.

The forward jaw positioning capabilities provided by the integrated system may optimize airway support by preventing the tongue from falling backward toward the throat or pressing against the soft palate in a manner that could obstruct airflow during sleep. The asymmetrical inner ridge may maintain the lower jaw in a position that creates adequate space within the oral cavity for unobstructed breathing while preventing the relaxation of jaw muscles that might otherwise allow the lower jaw to move backward into positions that compromise airway patency. The position adjusters (108; 114) may enable the apparatus to be configured for individual anatomical requirements while ensuring that the forward jaw positioning is maintained across the range of adjustments that may be needed to accommodate different users or changing anatomical conditions over time.

The user comfort aspects of the integrated system may be enhanced through the coordinated operation of the position adjusters (108; 114) and the asymmetrical ridge design that distribute positioning forces across multiple contact points and structural elements. The position adjusters (108; 114) may accommodate natural jaw movements by allowing controlled displacement within the protuberance and channel system, reducing the likelihood of binding or excessive stress on individual components during movement cycles. The asymmetrical ridge design may provide comfortable engagement with the dental structures by distributing contact forces across the height differential between the inner ridge and the outer ridge, preventing pressure concentrations that might cause discomfort during extended wear periods. In some cases, the integration system may allow for gradual adaptation to the forward jaw positioning by enabling incremental adjustments through the position adjuster system while maintaining the structural characteristics needed for effective airway support throughout the adaptation process.

### Disclaimers

Terms such as “top,” “bottom,” “front,” “rear,” “above,” “below” and the like as used in this disclosure should be understood as referring to an arbitrary frame of reference, rather than to the ordinary gravitational frame of reference. Thus, for example, an item disposed above another item may be located above or below the other item along a vertical, horizontal or diagonal direction; and an item disposed below another item may be located below or above the other item along a vertical, horizontal or diagonal direction. Some frames of references and the position of claimed structure relative to those frames of reference may be gleaned from the appended drawings. Yet, it should be understood that for some embodiments, the detection from devices disclosed herein may not be restricted to being accomplished from any particular position shown, whether the device is above, below, or on one side of the user being monitored.

Those of skill in the art would appreciate that various components and blocks may be arranged differently (e.g., arranged in a different order, or partitioned in a different way) all without departing from the scope of the subject technology. The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein, but are to be accorded the full scope consistent with the language of claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. An apparatus, comprising:
a top tray configured to be inserted into a mouth of a user, the top tray comprising an upper teeth channel, wherein the upper teeth channel is configured to fit around or conform to two or more upper teeth of the user;
a top position adjuster coupled to an underside of the top tray;
a bottom tray configured to be inserted into the mouth of the user, comprising:
a lower teeth channel, wherein the lower teeth channel is configured to fit around or conform to two or more lower teeth of the user; and
an outer ridge on a labial side of the lower teeth channel;
an inner ridge on a lingual side of the lower teeth channel, wherein the inner ridge is recessed from a rear edge of the bottom tray, wherein the inner ridge is asymmetrical to the outer ridge such that a height of the inner ridge is larger than a height of the outer ridge, the height differential between the inner ridge and the outer ridge creating a structural bias that encourages forward positioning of the lower jaw during use; and
a bottom position adjuster coupled to the bottom tray and positioned to interface with the top position adjuster.

2. The apparatus of claim 1, further comprising a recess on an underside of the top position adjuster, wherein only one lateral side of the recess is bounded by a wall on an outer edge of the top position adjuster.

3. The apparatus of claim 2, wherein the recess is unbounded on an interior, lingual edge of the top position adjuster.

4. The apparatus of claim 1, wherein:
the top tray or the bottom tray is a unibody;
the top tray and/or the bottom tray comprises a thermoplastic material that has a molding temperature less than or equal to 200° F.

5. The apparatus of claim 1, further comprising a centralized support element projecting from a bottom surface of the top position adjuster, wherein the centralized support element includes a curved, bottom edge.

6. The apparatus of claim 5, wherein a bottom surface of the centralized support element is spaced from a top surface of the bottom position adjuster and configured to maintain an open airway between an upper mandible tray and a lower mandible tray as the user wears the upper mandible tray and the lower mandible tray.

7. An apparatus, comprising:
a top tray configured to be inserted into a mouth of a user, the top tray comprising:
an upper teeth channel configured to fit around upper teeth of the user; and
a first set of fastener sides on an underside of the top tray;
a top position adjuster, comprising:
a second set of fastener sides complimentary with the first set of fastener sides, wherein the top tray and the top position adjuster are joined by the first set of fastener sides and the second set of fastener sides to form an upper mandible tray;
a set of position aligners on an underside of the top position adjuster, wherein the set of position aligners comprises:
a first row of aligners comprising:
a first protrusion comprising a first peg, a first pin, or a first dowel; and
a second protrusion comprising a second peg, a second pin, or a second dowel; and
a second row of aligners comprising:
a third protrusion comprising a third peg, a third pin, or a third dowel; and
a fourth protrusion comprising a fourth post, a fourth peg, a fourth pin, or a fourth dowel; and
a recess on the underside of the top position adjuster, wherein only one lateral side of the recess is bounded by a wall on an outer edge of the top position adjuster; and
a bottom tray configured to be inserted into the mouth of the user, the bottom tray comprising:
a lower teeth channel configured to fit around lower teeth of the user;
an outer ridge on a labial side of the lower teeth channel;
an inner ridge on a lingual side of the lower teeth channel, wherein the inner ridge is recessed from a rear edge of the bottom tray, wherein the inner ridge is asymmetrical to the outer ridge such that a height of the inner ridge is larger than a height of the outer ridge, the height differential between the inner ridge and the outer ridge creating a structural bias that encourages forward positioning of the lower jaw during use; and
a third set of fastener sides on a top side of the bottom tray; and
a bottom position adjuster, comprising:
a fourth set of fastener sides complimentary with the third set of fastener sides, wherein the bottom tray and the bottom position adjuster are joined by the third set of fastener sides and the fourth set of fastener sides to form a lower mandible tray;
a set of openings corresponding to the set of position aligners and positioned on a top side of the bottom position adjuster, the set of openings comprising:
a first cavity or a first channel configured to receive one of the first row of aligners or the second row of aligners;
a second cavity or a second channel configured to receive one of the first row of aligners or the second row of aligners; and
a third cavity or a third channel configured to receive one of the first row of aligners or the second row of aligners, wherein the set of position aligners and the set of openings are configured to connect to adjust an alignment of the upper mandible tray and the lower mandible tray laterally and longitudinally;
wherein, the apparatus is configured so that as the user wears the upper mandible tray and the lower mandible tray, the set of position aligners is positioned to fit into the set of openings to connect the upper mandible tray and the lower mandible tray in a first position; the set of position aligners is positioned to fit into the set of openings to connect the upper mandible tray and the lower mandible tray in a second position that is different from the first position;
wherein the first position and the second position are laterally and longitudinally different; and wherein the upper mandible tray and the lower mandible tray are configured to prevent occlusion or obstruction of an airway of the user.

8. The apparatus of claim 7, wherein the recess is unbounded on an interior, lingual edge of the top position adjuster.

9. The apparatus of claim 7, wherein:
the top tray comprises a first compressible barrier configured to be positioned between the upper teeth and the top position adjuster as the user wears the upper mandible tray; or
the bottom tray comprises a second compressible barrier configured to be positioned between the lower teeth and the bottom position adjuster as the user wears the lower mandible tray.

10. The apparatus of claim 7, wherein:
the top tray comprises a remoldable polymer, wherein the remoldable polymer retains shape at a temperature up to 110° F. and changes shape at a temperature between 110° F. and 200° F.;
the bottom tray comprises the remoldable polymer;
the top position adjuster comprises a rigid polymer, wherein the rigid polymer retains shape at temperatures between 110° F. and 200° F.; or
the bottom position adjuster comprises the rigid polymer.

11. The apparatus of claim 10, wherein:
the remoldable polymer comprises ethylene-vinyl acetate; or
the rigid polymer comprises polyurethane plastic.

12. The apparatus of claim 7, wherein:
the first set of fastener sides, the second set of fastener sides, the third set of fastener sides and the fourth set of fastener sides each comprise a hook-and-catch snap joint.

13. An apparatus comprising:
a top tray configured to be inserted into a mouth of a user, the top tray comprising:
an upper teeth channel including a plurality of crests and valleys in a wave pattern, configured to fit around upper teeth of the user; and
a first set of fastener sides on an underside of the top tray;
a top position adjuster, comprising:
a second set of fastener sides complimentary with the first set of fastener sides, wherein the top tray and the top position adjuster are joined by the first set of fastener sides and the second set of fastener sides to form an upper mandible tray;
a set of position aligners on an underside of the top position adjuster, wherein the set of position aligners comprises:
a first row of aligners comprising a first peg and a second peg; and
a second row of aligners comprising a third peg and a fourth peg; and
a recess on the underside of the top position adjuster, wherein only one lateral side of the recess is bounded by a wall on an outer edge of the top position adjuster; and
a bottom tray configured to be inserted into the mouth of the user, the bottom tray comprising:
a lower teeth channel configured to fit around lower teeth of the user;
an outer ridge on a labial side of the lower teeth channel;
an inner ridge on a lingual side of the lower teeth channel, wherein the inner ridge is recessed from a rear edge of the bottom tray, wherein the inner ridge is asymmetrical to the outer ridge such that a height of the inner ridge is larger than a height of the outer ridge, the height differential between the inner ridge and the outer ridge creating a structural bias that encourages forward positioning of the lower jaw during use; and
a third set of fastener sides on a top side of the bottom tray; and
a bottom position adjuster configured such that a front of the bottom position adjuster is positioned over a bottom incisor of the user as the user wears a lower mandible tray, the bottom position adjuster comprising:
a fourth set of fastener sides complimentary with the third set of fastener sides, wherein the bottom tray and the bottom position adjuster are joined by the third set of fastener sides and the fourth set of fastener sides to form the lower mandible tray;
a set of openings corresponding to the set of position aligners and positioned on a top side of the bottom position adjuster, the set of openings comprising:
a first channel configured to be aligned perpendicularly to a row of bottom teeth of the user and configured to receive one of the first row of aligners or the second row of aligners;
a second channel configured to be aligned perpendicularly to the row of bottom teeth of the user and configured to receive one of the first row of aligners or the second row of aligners; and
a third channel configured to be aligned perpendicularly to the row of bottom teeth of the user and configured to receive one of the first row of aligners or the second row of aligners, wherein the set of position aligners and the set of openings are configured to connect to adjust an alignment of the upper mandible tray and the lower mandible tray laterally and longitudinally;
wherein: the first channel, second channel and third channel are each curved towards the front of the bottom position adjuster or each comprise a bend, wherein an outside of the bend faces towards the front of the bottom position adjuster;
wherein the apparatus is configured so that as the user wears the upper mandible tray and the lower mandible tray, the set of position aligners is positioned to fit into the set of openings to connect the upper mandible tray and the lower mandible tray in a first position; the set of position aligners is positioned to fit into the set of openings to connect the upper mandible tray and the lower mandible tray in a second position that is different from the first position;
wherein the first position and the second position are laterally and longitudinally different; and wherein the upper mandible tray and the lower mandible tray are configured to prevent occlusion or obstruction of an airway of the user.

14. The apparatus of claim 13, wherein the recess is unbounded on an interior, lingual edge of the top position adjuster.

15. The apparatus of claim 13, wherein the plurality of valleys are configured to provide space for gum tissue expansion and the plurality of crests are configured to maintain secure positioning of the top tray and bottom tray around the user's teeth.

16. The apparatus of claim 13, wherein the wave pattern reduces overall material volume while maintaining structural integrity of the apparatus.

17. The apparatus of claim 13, wherein the first channel, second channel, and third channel are arranged perpendicular to the row of the user's bottom teeth and include cavities positioned within the channels to receive the set of position aligners.

18. The apparatus of claim 13, wherein the set of position aligners is disposed in the recess of the top position adjuster.

19. The apparatus of claim 17, wherein the set of position aligners comprise pegs configured to engage with the cavities to provide controlled positioning adjustment in lateral and longitudinal directions.

20. The apparatus of claim 13, further comprising a centralized support element positioned between the top position adjuster and the bottom position adjuster, wherein:
the centralized support element includes a curved, bottom edge;
